(19) United States
(12) Patent Application Publication
(10) Pub. No.: US 2011/0014597 A1
(43) Pub. Date: Jan. 20, 2011

(54) Perfusable Bioreactor for the Production and/or Cultivation of a Human or Animal Blood Vessel and/or a Human or Animal Tissue

(75) Inventor: Bernhard Frerich, Rostock (DE)

Correspondence Address: LERNER GREENBERG STEMER LLP, P O BOX 2480, HOLLYWOOD, FL, 33022-2480, US

(73) Assignee: NOVATISSUE GMBH, LEIPZIG (DE)

(21) Appl. No.: 12/934,844

(22) PCT Filed: Mar. 23, 2009
(86) PCT No.: PCT/EP2009/002110

§ 371 Date: Sep. 27, 2010

(30) Foreign Application Priority Data
Mar. 25, 2008 (DE) 10 2008 015 633.7

(51) Int. Cl. C12Q 1/02; C12M 3/00; C12N 5/00; A01N 1/00

(52) U.S. Cl. 435/1.2; 435/293.1; 435/395; 435/29; 435/284.1

(57) ABSTRACT

A bioreactor for the production and/or cultivation of a human or animal blood vessel and/or a human or animal tissue contains at least one tubular base body having two front sides. The bioreactor further has a reclosable liquid-tight opening which is arranged on one front side, an inner space, a reactor wall, at least one inlet and at least one outlet for a liquid medium and a mounting for the construct to be introduced.

The invention relates to a perfusable bioreactor for producing and/or cultivating human or animal tissue or tissue equivalents, preferably in conjunction with a human or animal blood vessel or blood-vessel equivalent.

Within the scope of the invention, “structures” are artificially produced three-dimensional tissue equivalents containing living cells, more particularly combinations of structures and living cells, possibly also combined with matrix factors, in a three-dimensional matrix.

Within the scope of the invention, the designations blood-vessel equivalents and blood-vessel-wall equivalents are used analogously to this definition.

Many different types of perfusable bioreactors have been developed to date for in vitro production of tissues and blood vessels. However, the focus up until now has mainly been on producing bioreactors with rigid walls. It is for this reason that many loads and influences on the tissue growing in vitro are not physiological. However, it is precisely mechanical loads that have an influence on the tissue growth in vivo and in vitro that should not be underestimated and should likewise be modeled.

This problem plays a role in particular in soft tissue and blood vessels. The provision or production of food and perfusion through blood vessels is a problem that in essence is yet to be solved in tissue engineering, i.e. cell and tissue cultivation. Therefore, there is a need for bioreactors in which a supplying blood vessel or a blood-vessel wall can be cultivated in conjunction with any tissue (“vessel-tissue unit”) and which moreover satisfies the physical/mechanical requirements of soft-tissue and/or vascular/microvascular engineering. This need exists beyond the confines of regenerative medicine for testing pharmacological substances as well, more particularly for those in which the interactions between vessels and tissues play a role, e.g. for circulatory diseases or obesity. Such a model would also be desirable for basic research in respect of such diseases and this may in certain circumstances afford the possibility of reducing animal testing.

Although previous bioreactors have already implemented pulsating perfusions, which are intended to simulate blood pressure, more particularly to condition artificial vessel structures to the blood pressure forces in vivo, said pulsating perfusions often are not physiological or are reflected in a non-physiological fashion in the surroundings of a rigid bioreactor wall, which can also lead to the destruction of the cells in the reactor. Thus, the provision of physiological tissue compliance (elasticity of the tissue) in the three-dimensional environment is required, and the previous systems do not allow this.

Furthermore, such a bioreactor must offer the option of being able to assess the development of such a blood vessel network or the development of the tissue/tissue equivalent both functionally and morphologically, continuously with non-invasive methods where possible. Many of the previous bioreactors do not offer the possibility of carrying out comprehensive optical or functional monitoring with a plurality of modalities, and at the same time measuring the pressure and the flow of the perfusion. Thus, up until now, it has been difficult or even impossible to visualize or monitor processes such as the vascularization of a tissue starting from a blood-vessel wall or a blood-vessel-wall equivalent produced by means of engineering using optical instruments, e.g. laser scanning microscopy (confocal microscope), or functionally using ESR (electron spin resonance) spectroscopy or benchtop MRI (magnetic resonance imaging) scanners because they cannot be introduced into the tunnel of these instruments due to their size. However, this is necessary to be able to assess physical, hydrodynamic cultivation parameters and growth factor effects on the development of a functional vessel network in vitro.

A specific problem when including a vessel, a vessel structure or a vessel wall, a vessel structure in a bioreactor with small dimensions suitable for the aforementioned examinations is the controlled provision of a relative high pressure in the vessel compartment—at least during the pulsation (systole)—compared to the “interstitium”, as in natural vessels and tissues. This is particularly challenging if a vessel-wall equivalent is intended to be clamped because the surface thereof should remain observable at the same time. Due to the fragility of such structures, the problem consists of ensuring a seal that allows an at least temporary pressure increase in the vessel compartment (as a result of the pulse in the case of a pulsating perfusion). A model permitting this in limited space can be used in many different questions from the fields of regeneration and tissue engineering, angiogenesis, circulation and metabolism research, oncology (metastasis) and many more.

An object of the invention is to provide a bioreactor that overcomes the disadvantages of the prior art.

The object of the invention is achieved by the features of claim 1.

More particularly, what is essential to the invention is that the bioreactor has large proportions of elastic walls, has a tubular design and that a blood vessel, a blood-vessel wall or a corresponding structure (“blood-vessel equivalent”) can be introduced into the interior thereof such that it is arranged in the longitudinal axis of the bioreactor and can be perfused with relatively high pressure compared to the surrounding pressure (in the bioreactor) and moreover that transparent viewing windows offer the possibility of observing said blood vessel, blood-vessel wall or corresponding structure using optical magnification instruments, e.g. laser scanning microscopes.

All connectors and openings for introducing and manipulating tissues, structures or structure-cell combinations are introduced from the head faces. The overall diameter preferably does not exceed 17 mm, particularly preferably 13 mm, and so the bioreactor can be examined during operation thereof in the tunnel of benchtop MRI and ESR scanners. Perfusion is brought about by means of a self-regulating perfusion system, which transports the perfusion medium and allows different perfusion modes to be set (e.g. pulsation, frequency, etc.).

There are two variants: In variant 1, the blood-vessel wall or the blood-vessel equivalent is arranged with its (endothelial) inner side open to the viewing pane. The perfusable chamber thus is formed by the blood-vessel wall/blood-vessel-wall equivalent, by the viewing pane (which can likewise be produced from elastic material) and by a circumferential rigid pressure surface. The chamber is sealed with respect to the remaining chamber volume by virtue of the fact that the blood-vessel wall/blood-vessel-wall equivalent is pressed against the circumferential pressure surface by an elastic shaped body (e.g. elastic foam). The pressure in the region of the circumferential pressure surface can be supported by virtue of the fact that a second frame congruent to the pressure surface is placed on the lower surface of the blood-vessel wall/blood-vessel-wall equivalent and, furthermore, by virtue of the fact that the elastic shaped body has a biphasic design, i.e. it has a less elastic circumferential wall, which supports the blood-vessel wall/blood-vessel-wall equivalent in the region of the circumferential pressure surface, and a more elastic core which favors the pressure-dependent deflections of the central region of the blood-vessel wall/blood-vessel-wall equivalent. These measures create a circumferential seal, which, although not completely liquid-tight, provides a seal to the extent that, whilst sparing the blood-vessel wall/blood-vessel-wall equivalent, there is a temporary, measurable pressure increase (as much as up to physiological and pathological systole values) in the perfusable chamber during the pulse phase.

This basic design offers the option of placing any tissue or tissue equivalent between blood-vessel wall/blood-vessel-wall equivalent and elastic shaped body, into which tissue or tissue equivalent small blood vessels can sprout from the blood-vessel wall/blood-vessel-wall equivalent, which is intended to be observed using the aforementioned imaging and measurement methods. It is also possible for the entire remaining bioreactor cavity to be filled with such a tissue or tissue equivalent instead of with the elastic shaped body such that the elastic bioreactor wall alone presses the blood-vessel wall/blood-vessel-wall equivalent against the circumferential pressure surface as a result of its pressure and hence brings about the seal.

The perfusion medium can leave the perfusable chamber via the aforementioned outlet and, additionally, through the tissue (perfusion in the actual sense of the word, e.g. through grown capillaries or artificially opened channels in the tissue piece) into the remaining volume of the bioreactor filled by foam/elastic shaped material, from where it leaves the bioreactor via an additional outlet. A tissue unit can be simulated functionally and anatomically therewith, which tissue unit consists of a supplying blood vessel and any attached, supplied tissue.

Thus, for example, pulsatile perfusion is transmitted through the tissue against the foam or the elastic wall, and mechanical or hydrodynamic loads can act on the tissue. The essential difference from earlier solutions lies in the fact that these perfusion dynamics occur in elastic surroundings and in that setting the elasticity of the chamber wall enables the compliance of natural blood vessels and tissue in physiological and pathological situations to be modeled.

The connectors for monitoring systems can be integrated into the wall and/or the circumferential pressure surface. The monitoring (observing and controlling) is brought about by means of a probe system, which monitors matter concentrations and physical or chemical variables such as e.g. O₂ and CO₂ concentration, pressure in the chamber and in the remaining bioreactor, partial pressure of oxygen, pH, flow velocity and temperature. The stretch of the elastic walls can be monitored by strain gauges. The monitoring moreover actively contributes to regulating the growth conditions in the bioreactor system, because it is included in a closed-loop control as a sensor system.

In the second variant of the bioreactor according to the invention, the perfusable chamber consists of a blood vessel or blood-vessel equivalent, which is guided along the viewing pane and obtains an inlet and outlet by means of two connectors. This likewise allows observation using CLSM, MRI (magnetic resonance imaging scanner) or ESR (electron spin resonance equipment), but not by directly viewing the endothelial surface. Rather than having a separate viewing pane, the entire elastic component of the bioreactor wall can also be produced from a clearly transparent elastic material.

It follows that the shape and arrangement of all components allow the production of particularly small or thin bioreactors, which are bound by spatially limiting prescriptions in specific application cases, e.g. under a laser scanning microscope or in the tunnel of ESR equipment and benchtop MRIs. All advantages of the invention just listed above thus clearly contribute to improving previous bioreactor systems, the growth conditions in bioreactors and the quality of cultivated tissue structures, and have positive effects on tissue engineering in general and in particular.

The bioreactor can be designed for single use.

### EXPLANATION OF THE INVENTION USING EXEMPLARY EMBODIMENTS

### Example 1

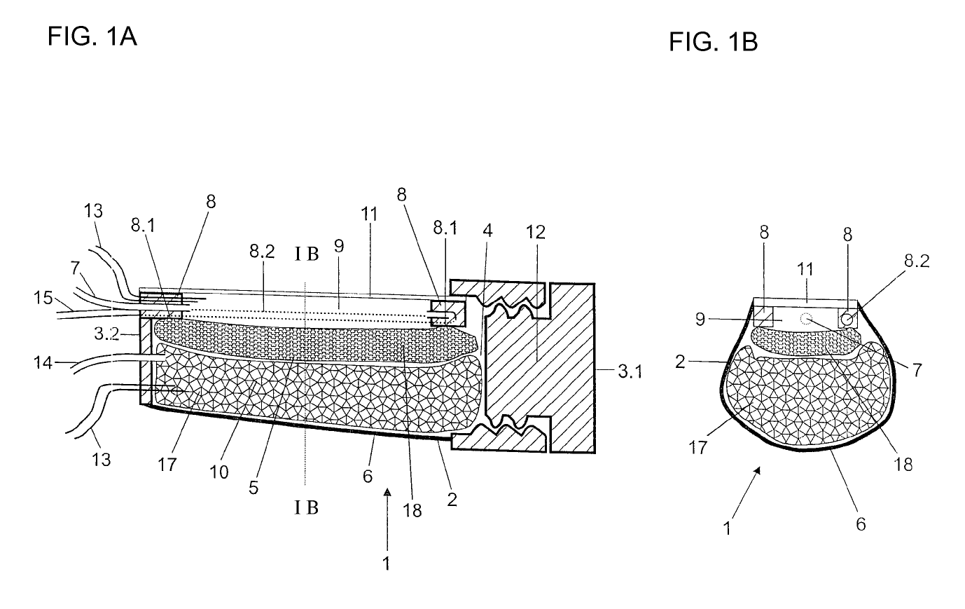

### Bioreactor According to FIGS. 1a and 1b

The bioreactor 1 is used for producing and/or cultivating a human or animal blood vessel and/or a human or animal tissue (vessel-tissue piece). The bioreactor 1 has a tubular, hollow base body 2 with two end faces 3.1 and 3.2.

A resealable, liquid-tight opening 4 for loading and construction is arranged at the end face 3.1, which opening more particularly serves for introducing the structure 18 into the interior 5 of the reactor. The opening 4 is designed as a conventional, liquid-tight screw cap 12.

Arranged in the interior 5 there is a perfusable pressure chamber (perfusable chamber) 9, which simultaneously is part of the interior 5, is arranged approximately parallel to the reactor longitudinal axis of the reactor 1 and is open toward the reactor axis. In that part of the perfusable pressure chamber (perfusable chamber) 9, which is arranged in the region of the end face 3.2, the inlet 7 opens into the pressure chamber (perfusable chamber) 9. Probes (e.g. pressure, flow, pH) 13 of the monitoring system are also arranged in this region and project into the pressure chamber (perfusable chamber) 9.

The pressure chamber (perfusable chamber) 9 comprises a circumferential pressure surface 8.1 of the support 8, against which the structure (tissue piece) 18, in this case a vessel-wall equivalent, is pressed in liquid-tight fashion. The structure 18 therefore forms a perfusable separation wall between the pressure chamber (perfusable chamber) 9 and the structure chamber 10, with the structure being pressed against the pressure surface 8.1 in the process.

Part of the pressure chamber (perfusable chamber) 9 forms the monitoring window 11, which is designed as a transparent viewing pane.

The interior 5 moreover contains a structure chamber 10, which is arranged approximately parallel to the reactor longitudinal axis and is open toward the reactor axis and closed by the structure in the filled state, as illustrated in FIG. 1.

The outlet (outlet out of the reactor space) 14 opens into the structure chamber 10 in the part of the structure chamber 10 arranged in the region of the end face 3.2. The remaining cavity of the structure chamber 10, i.e. the space that is not filled by the structure is, as illustrated in FIG. 1, filled by an elastic shaped part (elastic shaped body) 17.

A part of the reactor wall 6 that is part of the structure chamber 10 consists of an elastic material, for example elastic silicone foil.

The tubular base body 2 is dimensioned such that it can be inserted into the opening of an ESR or benchtop MRI scanner. Hence, the end faces 3.1 and 3.2 each do not have a diameter exceeding 13 mm (or 17 mm for ESR).

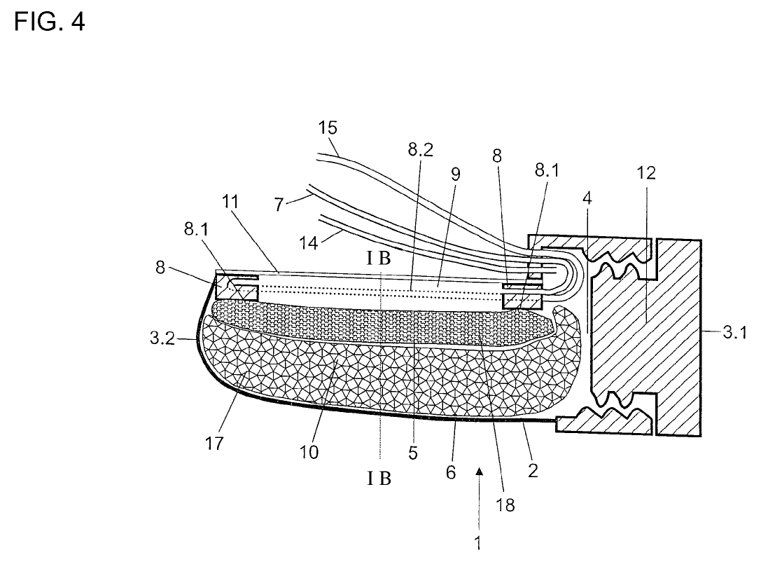

In the state illustrated in FIG. 1, a culture medium, blood or the like is introduced with high pressure into the pressure chamber 9 in the bioreactor 1 via the inlet 7. Part of this liquid medium flows through the perfusable structure and thus reaches the region of the structure chamber 10. Liquid medium is guided out of the bioreactor via the outlets 14 and 15. The liquid medium is then supplied to a pressure-generating unit, for example a peristaltic pump. The pressure-generating unit can also generate pulsating inflows, which are then present in the pressure chamber (perfusable chamber) 9 in a pulsating state. This brings about an at least partial deflection of the structure toward the structure chamber 10 and hence also stretching of the elastic reactor wall 6 on the outside as a result of displacing the contents of the structure chamber 10. Other arrangements with the same basic principle are also possible (FIG. 4).

### Example 2

### Operation of the Partly Elastic Bioreactor

A prepopulated, flat vessel-wall equivalent 18, e.g. consisting of a structure with mesenchymal stem cells and endothelial cells for simulating a vessel wall can now be cut to the size of the circumferential pressure surface 8.1 and be placed flat on the latter (the endothelial side facing the viewing plate 11). This produces a gap-shaped cavity 9 between the glass plate 11 and tissue equivalent, which cavity corresponds to the perfusable chamber volume and can be observed directly from above through the glass plate 11. The side of the tissue facing the viewing plate 11 corresponds to the inner side of a blood-vessel wall in this model. The viewing plate 11 allows direct optical monitoring (e.g. by means of microscopy, fluorescence microscopy, laser scanning microscopy, etc.) of the developing surface of the vessel wall on the boundary surface to the perfusion chamber 9 and also non-invasive functional monitoring (e.g. by ESR spectroscopy, MRI) of the vessel-wall equivalent during the reactor operation.

A tissue equivalent 19, e.g. a scaffold with mesenchymal stem cells from a target tissue, e.g. muscle, fatty tissue or bone, is placed on the lower surface of the vessel-wall equivalent 18. The remaining cavity of the bioreactor 1 is filled with an elastic foam. In the case of a pulsating or other pressure perfusion, the tissue equivalent/vessel-wall equivalent is subjected to physiological or pathological pressures and deviations as necessary, which can be measured by pressure measurement systems in the chamber interior.

### Example 3

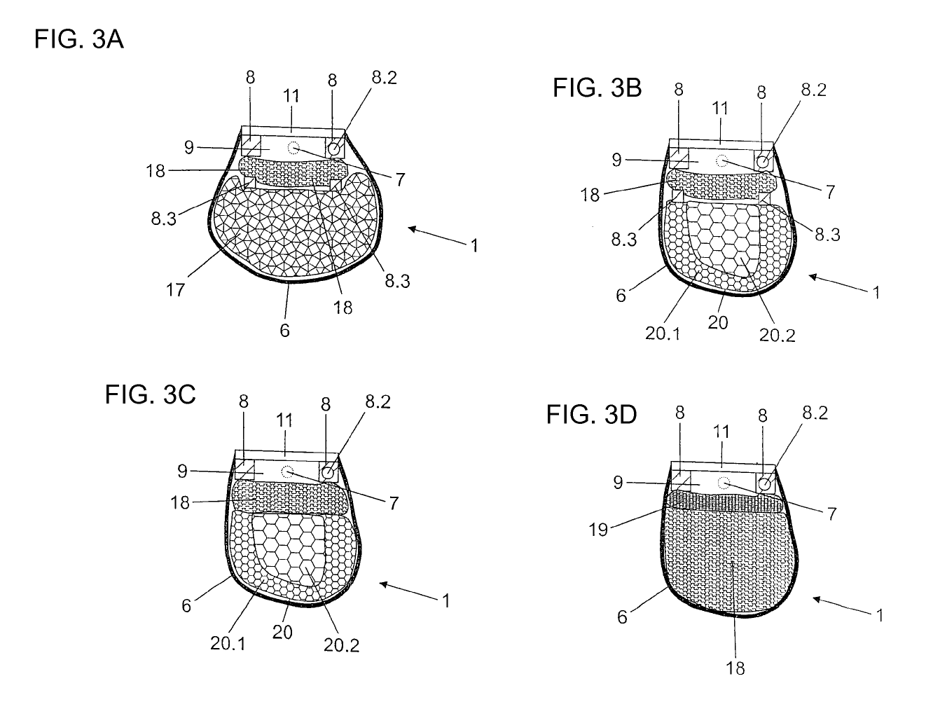

### Measures for Sealing the Circumferential Pressure Surface (FIG. 3)

An elastic fixation device (e.g. a formed piece of foam) 17 formed appropriately for the bioreactor interior is introduced into the bioreactor interior and pushes the tissue piece 18 against the frame 8, as a result of which the perfusable chamber volume 9 is sealed. The foam thus fills the cavity between the tissue piece 18 and the flexible wall 6 and at the same time fixes the tissue piece 18 on the frame 8.

Here, another fixed or elastic plastics frame (frame additionally placed therebetween) 8.3 can in addition be placed between tissue piece 18 and foam 17 within the extent of the frame on the viewing plate 11, and so the edge of the tissue piece 18 is fixed between the two frames (FIG. 3a).

The diameter of the bioreactor interior can be reduced as an alternative to the foam insert in order to fix the tissue piece directly between frame and elastic wall, or the tissue piece or tissue equivalent 18 with a vessel wall or a vessel-wall equivalent 19 can be embodied to be so large as to fill the cavity completely, that is to say without foam (FIG. 3d: cross section through bioreactor 1 without shaped body, with vessel or vessel equivalent and attached tissue or tissue equivalent, completely filling the bioreactor space).

The compliance can be further supplemented and set by variants of the elastic shaped body (foam). Thus, for example, a biphasic elastic shaped body/foam 20 can be used, in which the edge (solid zone) 20.1 is less elastic than the center (zone with high elasticity) 20.2. What this achieves is that the pulsating perfusion deflects the tissue piece outwards more easily in the center and fixes and seals said tissue piece on the edge in an improved fashion (FIG. 3b with biphasic shaped body and frame placed therebetween and FIG. 3c with biphasic shaped body without frame placed therebetween).

### Example 4

### Additional Integration of a Device for Regulating the Elasticity of the Perfusable Chamber

In order to further adapt the elasticity (“compliance”) of the perfusable chamber, the viewing plate can be modified as follows: for this purpose, it is provided with a central recess matched to the size of the frame, and a second support plate is produced with an identical recess. A highly elastic membrane can now be bonded between the two support plates and from here on in acts as a new portion of the wall and as a regulator of the compliance by extending outward during each pulse and tightening again thereafter. This membrane is preferably transparent so that the perfused tissue surface can be observed and examined under a microscope therethrough. Hence the entire chamber wall is made up of elastic material with the exception of the fixed frame.

### Example 5

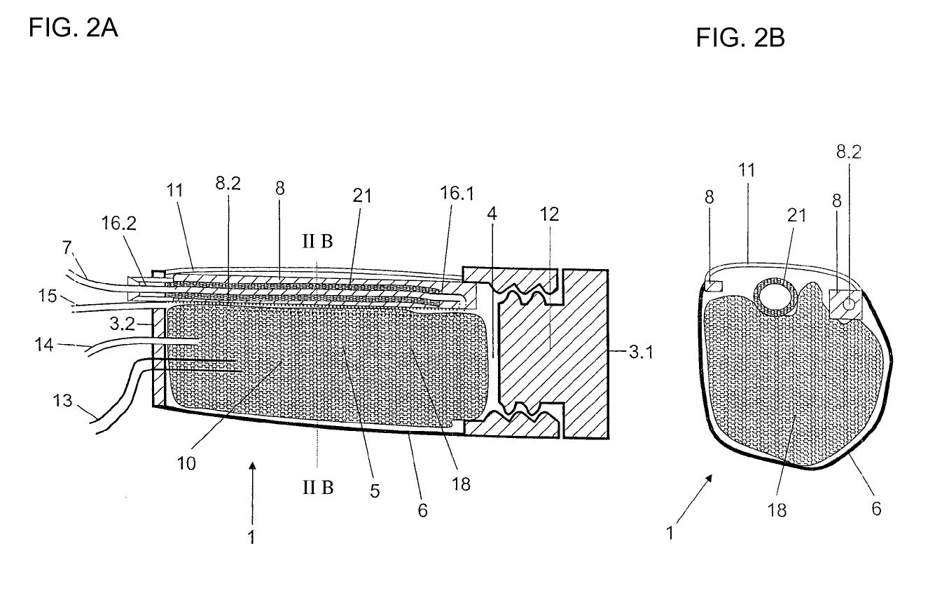

### Variant with a Tubular Vessel (FIGS. 2a and 2b)

One embodiment variant consists of there not being a perfusable chamber as such, but rather a thin support 8 being integrated into the chamber wall and connecting the two end faces, at the ends of which connectors are attached in each case, which serve for connecting a blood vessel or a blood-vessel equivalent 21. The connector at the inlet 7 must be embodied such that the vessel/vessel equivalent 21 can be introduced in a sterile fashion through the large opening 4 and can be coupled to the end face 3.2. For this purpose, the end face 3.2 is provided with a smaller opening with a flange, through which a coupling 16.2 with a tube clip can be introduced from the outside, onto which the vessel/vessel equivalent 21 is fixed. This coupling 16.2 is attached, e.g. according to the Luer-lock principle, to the flange in a liquid-tight fashion and fixes the vessel/vessel equivalent 21. Thereafter the vessel/vessel equivalent 21 is attached to the connector 16.1 (e.g. tube clip). The line 8.3 for the outlet 15 is guided in the frame 8 as in the first-described variant, or along it. Hence a tubular structure is perfused; all other features of the chamber are maintained. Since the perfusion surface is no longer visible, optical methods for monitoring have been made more difficult than in the first variant. However, this allows the production or simulation of a blood vessel by means of tissue engineering, which vessel is in direct contact with a supplied tissue section (structure, tissue piece) 18. By way of example, this affords the possibility of examining the conditions under which sproutings (small blood vessels) grow into the attached tissue (structure, tissue piece) 18 from the central vessel. If human or animal blood vessels or tissue are taken instead of structures produced by tissue engineering, it is also possible to examine physiological or pathological processes in vitro that until now were reserved for animal testing. This preferably holds true for pathological and physiological processes in vessels or the circulatory system, for obesity research and for testing pharmacological substances in which the interactions between blood vessels and tissue play a role.

In the exemplary embodiment as per FIGS. 2a and 2b, the monitoring window 11 is designed as a transparent film.

### Example 6

### Connection and Operation of the Self-Regulating Pulsating Perfusion System

A self-regulating perfusion system operating in a pulsed fashion is connected to the bioreactor and is used for simulating physiological or experimental pressure conditions.

### Example 7

### Monitoring

A comprehensive monitoring system is connected using the connectors of the frame on the glass pane of the bioreactor and allows monitoring of important parameters (O₂, SpO₂, CO₂, pH, pressure, temperature, viscosity, flow velocities, etc.) in real time.

### Example 8

### Applications

Application options for the bioreactor according to the invention present themselves wherever the interactions between vessel and stroma or mesenchymal or other tissue play a role. These include many fields in addition to the previously sketched applications in regenerative medicine and tissue engineering. As sketched in the previous examples, the system can likewise be operated with natural explanted tissues and vessels, analogously to the tissue equivalents and artificial tissues. This results in a broad field of application. Said applications include, for example, basic-research oriented examinations, in particular in the research of circulatory diseases, and also many metabolic disorders, such as e.g. obesity, in which the interaction between vessels and fat cells plays an important role. Furthermore, it can be useful as a metastasis model in oncological research. Questions in respect of wound healing can be answered therewith, and it can also be used as an angiogenesis model in basic research. A substantial branch is also the application thereof in testing pharmacological agents, e.g. testing the transfer of pharmacological agents into the interstitium or other questions.

Here, and in other applications, it can also be used as a replacement for animal testing.

1-39. (canceled)

40. A perfusable bioreactor for producing or cultivating a human blood vessel, an animal blood vessel, a human tissue or an animal tissue, the bioreactor comprising:
a tubular base body containing:
two end faces having a resealable, liquid-tight opening formed in one of said end faces;
an interior;
a reactor wall;
at least one inlet and a first outlet for a liquid medium;
a support for a structure to be introduced;
a perfusable pressure chamber disposed parallel to a reactor longitudinal axis and disposed in said interior;
a second outlet, said second outlet and said inlet open into said perfusable pressure chamber and said perfusable pressure chamber is open toward a reactor axis;
a structure chamber disposed parallel to said reactor longitudinal axis and open to said reactor axis and disposed in said interior;
said first outlet opening into said structure chamber;
the structure introduced into said tubular base body can be disposed as a separation wall between said perfusable pressure chamber and said structure chamber; and
said reactor wall having at least one partial segment formed from an elastic material and being at least part of said structure chamber.

41. The bioreactor according to claim 40, wherein said partial segment has more than 50% of an inner surface of said reactor wall.

42. The bioreactor according to claim 40, further comprising a monitoring window, selected from the group consisting of transparent rigid monitoring windows and transparent elastic monitoring windows, and disposed in said reactor wall in a region of said perfusable pressure chamber.

43. The bioreactor according to claim 40, wherein the bioreactor is operable at a relatively high pressure, with a pressure acting in said perfusable pressure chamber being greater than that in said structure chamber.

44. The bioreactor according to claim 40, wherein said support has at least one pressure surface, and the structure can be pressed against said pressure surface.

45. The bioreactor according to claim 40, wherein said support has at least two connectors, and between said two connectors a tubular blood vessel, blood-vessel equivalent or structure for a blood vessel to be produced by means of tissue engineering can be clamped.

46. The bioreactor according to claim 40, wherein a physical pressure load regime that can be generated in said interior corresponds to a physical pressure load regime acting on a produced tissue, tissue equivalents, blood vessels or blood vessel networks in normal physiological or pathological conditions in a living human organism or a living animal organism.

47. The bioreactor according to claim 40, wherein an elasticity of said elastic material of said partial segment can be set such that a stretching of tissue or tissue equivalent in a sheath as a result of a perfusion pressure generated in said interior corresponds to a physiological or pathological values of a tissue compliance of a tissue to be produced.

48. The bioreactor according to claim 40, wherein said at least one inlet, said first and second outlets, and connectors for probes are guided into the perfusable bioreactor at said end faces.

49. The bioreactor according to claim 40, wherein the perfusable bioreactor has an overall diameter not exceeding 17 mm, and can be examined in a tunnel of electron spin resonance scanners and nuclear magnetic resonance scanners.

50. The bioreactor according to claim 40,
wherein at said resealable, liquid-tight opening tissues, blood vessels, equivalents and shaped bodies are introduced; and
further comprising a screw cap sealing said resealable, liquid-tight opening.

51. The bioreactor according to claim 42, wherein said perfusable pressure chamber and said monitoring window are dimensioned such that a vessel tissue piece can be observed using optical magnifying units, including fluorescence microscopes and confocal microscopes.

52. The bioreactor according to claim 40,
further comprising an elastic shaped body; and
wherein said tubular base body has a frame congruent to the structure and inserted between said elastic shaped body and the structure, said frame supports a bearing pressure of the structure in an edge region.

53. The bioreactor according to claim 52, wherein said elastic shaped body is formed from a biphasic elastic material and has central components, said biphasic elastic material has a higher elasticity in said central components than in an edge region, and so a deflection of a vessel tissue piece is amplified in said central component compared to said edge region as a result of a perfusion pressure and a support of a bearing pressure of a lateral edge region of the structure is achieved.

54. The bioreactor according to claim 42, further comprising a clamping device disposed in said interior, directly next to and along a region of said monitoring window and having two tube connectors, into said clamping device a blood vessel, a blood-vessel equivalent or a structure for a blood vessel to be produced by means of tissue engineering can be clamped between said two tube connectors such that the blood vessel, the blood-vessel equivalent or the structure for the blood vessel to be produced by means of tissue engineering can be observed from an outside through said monitoring window by means of optical equipment, including a confocal laser scanning microscopy.

55. The bioreactor according to claim 40, wherein one of said end faces has a further liquid-tight opening formed therein, by means of said further liquid-tight opening a blood vessel, a blood-vessel equivalent or the structure for a blood vessel to be produced by means of tissue engineering can be coupled to said inlet in sterile conditions, by means of a modified Luer-lock connector.

56. The bioreactor according to claim 40, wherein said perfusable pressure chamber, a blood vessel, a blood-vessel equivalent or a structure for a blood vessel to be produced by means of tissue engineering can be perfused by a culture medium, blood or a mixture of both.

57. The bioreactor according to claim 40, wherein the perfusable bioreactor can be operated as a unit in conjunction with a self-regulating, pulsating perfusion system.

58. The bioreactor according to claim 40, wherein the perfusable bioreactor does not require any metallic components.

59. The bioreactor according to claim 40, wherein mechanical loads of an order of physiological forces, including blood pressure values of an entire human vessel system, can be exerted on a tissue by means of perfusion dynamics.

60. The bioreactor according to claim 40, further comprising one of absorbable hollow fiber systems, non-absorbable hollow-fiber systems, line systems or frame structures disposed in said interior, by means of which a culture medium is distributed, which in turn feed a tissue or tissue equivalent to be produced.

61. The bioreactor according to claim 40, wherein said partial segment has more than 75% of an inner surface of said reactor wall.

62. The bioreactor according to claim 40, further comprising a reactor sheath made of a transparent material and disposed in a region of said pressure chamber.

63. The bioreactor according to claim 40, wherein the perfusable bioreactor has an overall diameter not exceeding 13 mm, and can be examined in a tunnel of electron spin resonance scanners and nuclear magnetic resonance scanners.

64. The bioreactor according to claim 40, wherein mechanical loads of an order of physiological forces, including blood pressure values of an entire human vessel system, can be exerted on a tissue by means of pulsating perfusion using a self-controlling perfusion system.

65. A method for producing one of human tissues, animal tissues, tissue equivalents, blood vessels or blood vessel networks, which comprises the steps of:
providing a perfusable bioreactor containing a tubular base body, the tubular base body containing:
two end faces with a resealable, liquid-tight opening formed in one of said end faces;
an interior;
a reactor wall;
at least one inlet and a first outlet for a liquid medium;
a support for a structure to be introduced;
a perfusable pressure chamber disposed parallel to a reactor longitudinal axis and disposed in the interior;
a second outlet, the second outlet and the inlet open into the perfusable pressure chamber and the perfusable pressure chamber is open toward a reactor axis;
a structure chamber disposed parallel to the reactor longitudinal axis and open to the reactor axis and disposed in the interior;
the first outlet opening into the structure chamber;
the structure introduced into the tubular base body can be disposed as a separation wall between the perfusable pressure chamber and the structure chamber;
the reactor wall having at least one partial segment formed from an elastic material and being at least part of the structure chamber; and
using the perfusable bioreactor for producing the human tissues, the animal tissues, the tissue equivalents, the blood vessels or the blood vessel networks.

66. The method according to claim 65, which further comprises producing a tissue equivalent by filling a cavity with a substrate prepopulated by human or animal, undifferentiated, predifferentiated or differentiated mesenchymal stem cells.

67. The method according to claim 66, which further comprises adding endothelial cells in addition to the mesenchymal stem cells.

68. The method according to claim 65, which further comprises adding a fibrin matrix.

69. A method for producing an artificial, supplying blood vessel system produced by means of tissue engineering, which comprises the steps of:
providing a perfusable bioreactor containing a tubular base body, the tubular base body containing:
two end faces with a resealable, liquid-tight opening formed in one of said end faces;
an interior;
a reactor wall;
at least one inlet and a first outlet for a liquid medium;
a support for a structure to be introduced;
a perfusable pressure chamber disposed parallel to a reactor longitudinal axis and disposed in the interior;
a second outlet, the second outlet and the inlet open into the perfusable pressure chamber and the perfusable pressure chamber is open toward a reactor axis;
a structure chamber disposed parallel to the reactor longitudinal axis and open to the reactor axis and disposed in the interior;
the first outlet opening into the structure chamber;
a structure introduced into the tubular base body can be disposed as a separation wall between the perfusable pressure chamber and the structure chamber;
the reactor wall having at least one partial segment formed from an elastic material and being at least part of the structure chamber;
introducing the structure into the interior of the perfusable bioreactor and a remaining cavity of the interior is filled with a liquid culture medium and perfused by a culture medium via the inlet and the first and second outlets.

70. The method according to claim 69, wherein it is at least in part performed by filling a cavity with a substrate prepopulated by human or animal cells.

71. The method according to claim 69, which further comprises adding the substrate or endothelial cells in a fibrin matrix.

72. The method according to claim 69, wherein a physical pressure load regime is exerted on the structure and corresponds to a physical pressure load regime acting on produced tissue in normal living conditions in a living human or animal organism, a liquid culture medium being introduced into the interior with high pressure and in a pulsating fashion via the inlet, and leaving the interior via at least one of the first and second outlets.

73. The method according to claim 69, wherein it is at least in part performed by filling a cavity with a substrate prepopulated by human mesenchymal stem cells or animal mesenchymal stem cells.

74. The method according to claim 73, which further comprises adding endothelial cells in addition to the mesenchymal stem cells.

75. A method for producing an artificial, supplying blood vessel system produced by means of tissue engineering, which develops vascular sproutings during a cultivation period and thereby takes over distributing a perfusion medium or supplying a surrounding tissue, which comprises the steps of:
providing a perfusable bioreactor containing a tubular base body, the tubular base body containing:
two end faces with a resealable, liquid-tight opening formed in one of said end faces;
an interior;
a reactor wall;
at least one inlet and a first outlet for a liquid medium;
a support for a structure to be introduced;
a perfusable pressure chamber disposed parallel to a reactor longitudinal axis and disposed in the interior;
a second outlet, the second outlet and the inlet open into the perfusable pressure chamber and the perfusable pressure chamber is open toward a reactor axis;
a structure chamber disposed parallel to the reactor longitudinal axis and open to the reactor axis and disposed in the interior;
the first outlet opening into the structure chamber;
a structure introduced into the tubular base body can be disposed as a separation wall between the perfusable pressure chamber and the structure chamber; and
the reactor wall having at least one partial segment formed from an elastic material and being at least part of the structure chamber;
using the perfusable bioreactor for producing the artificial, supplying blood vessel system.

76. A production method, which comprises the step of:
providing a perfusable bioreactor containing a tubular base body, the tubular base body containing:
two end faces with a resealable, liquid-tight opening formed in one of said end faces;
an interior;
a reactor wall;
at least one inlet and a first outlet for a liquid medium;
a support for a structure to be introduced;
a perfusable pressure chamber disposed parallel to a reactor longitudinal axis and disposed in the interior;
a second outlet, the second outlet and the inlet open into the perfusable pressure chamber and the perfusable pressure chamber is open toward a reactor axis;
a structure chamber disposed parallel to the reactor longitudinal axis and open to the reactor axis and disposed in the interior;
the first outlet opening into the structure chamber;
a structure introduced into the tubular base body can be disposed as a separation wall between the perfusable pressure chamber and the structure chamber; and
the reactor wall having at least one partial segment formed from an elastic material and being at least part of the structure chamber.

77. The method according to claim 76, which further comprises performing experimental applications in a field of tissue engineering and regenerative medicine with the perfusable bioreactor.

78. The method according to claim 76, which further comprises experimentally producing human or animal tissue, including vascularized soft tissue and a vessel replacement with a small lumen in the perfusable bioreactor.

79. The method according to claim 76, which further comprises producing human and animal tissues for clinical and therapeutic use in the perfusable bioreactor.

80. The method according to claim 76, which further comprises testing pharmacological substances in a field of circulation and obesity research in the perfusable bioreactor.

81. The method according to claim 76, which further comprises using the perfusable bioreactor as an angiogenesis model in wound healing and oncology.

82. The method according to claim 76, which further comprises using the perfusable bioreactor for oncological questions, including those relating to metastasis, and to testing pharmacological agents in respect of this.

83. The method according to claim 76, which further comprises using the perfusable bioreactor for examining the vascularization in pathological and physiological processes, including in vessel processes, in regeneration and in tissue engineering.

84. The method according to claim 76, which further comprises using the perfusable bioreactor to replace animal testing.
